(19) United States
(12) Patent Application Publication
(10) Pub. No.: US 2026/0325867 A1
(43) Pub. Date: Oct. 22, 2026

(54) INSTRUMENTS AND CORRESPONDING PROCEDURAL METHODS FOR PERFORMING PHOTOBIOMODULATION THERAPY

(71) Applicant: DENTROID PTY LTD, Australian Capital Territory (AU)

(72) Inventor: Laurence WALSH, Australian Capital Territory (AU)

(73) Assignee: DENTROID PTY LTD, Australian Capital Territory (AU)

(21) Appl. No.: 19/122,105

(22) PCT Filed: Oct. 20, 2023
(86) PCT No.: PCT/AU2023/051045

§ 371 Date: Apr. 17, 2025

(30) Foreign Application Priority Data
Oct. 20, 2022 (AU) 2022903093

(51) Int. Cl. A61N 5/06; A61C 13/15

(52) U.S. Cl. CPC A61N 5/062; A61C 19/003; A61N 2005/0606; A61N 2005/0627; A61N 2005/0632; A61N 2005/0644; A61N 2005/0652; A61N 2005/0659; A61N 2005/0663

(57) ABSTRACT

Embodiments relate to an instrument configured to perform photobiomodulation (PBM) therapy. The instrument includes a body having a light emitting head and a light source for emitting light through an emitting portion of the light emitting head. The light source is configured to simultaneously emit light at multiple peak wavelengths to perform photobiomodulation (PBM) therapy and wherein the multiple peak wavelengths fall within the spectral range of 615 nm to 1100 nm.

### FIELD OF INVENTION

The present invention relates to instruments and methods for performing photobiomodulation (PBM) therapy. More particularly, but by no means exclusively, embodiments relate to a handheld device configured to produce PBM dental analgesia within a patient's oral cavity for accompanying a dental procedure.

### BACKGROUND OF INVENTION

In clinical practice, there are many procedures that are uncomfortable for patients, and which require injections of local anaesthetic to allow them to be undertaken safely and effectively. Intense fear and anxiety are often associated with the injection procedure. Similar concerns apply when using a lancet on the skin, administering an injection (such as a vaccine) or placing an intravenous line. These can all be painful and anxiety provoking.

Additional examples include dental procedures such as placement of interdental wedges, orthodontic separators, and dental dam clamps, as well as scaling of teeth, filling of teeth, dental injections, and oral surgical procedures, such as frenectomy to treat tongue tie, or tooth removal.

Likewise, similar concerns of discomfort relating to skin or mucosal penetration exist for cosmetic procedures such as tattooing, tattoo removal, insertion of body piercings, hair removal using lasers or intense pulse lights, hair removal using electrolysis, injections of fillers and botulinum toxin, and needling of the skin. Finally, in the domestic setting, there are a range of self-performed procedures which are uncomfortable, including hair removal by plucking hair and waxing skin.

The use of visible red and near infrared diode lasers and Nd: YAG lasers to alter response thresholds and to induce analgesia has been described in the literature for over 25 years, and the use of diode laser units is commonplace in physiotherapy, veterinary medicine, and medicine, and to some extent also in dentistry.

In the patent literature, US 2009/0082759 (Pryor et. al.) describes a laser apparatus for the reduction of pain and inflammation caused by injections of the skin. Applications listed for the apparatus listed in this application include cosmetic injections, such as Botox or dermal filling, mesotherapy, immunization shots, etc. According to Pryor et al., laser application is provided either via an optical wand or flexible light emitting pad/bandage optically coupled to an optical fibre of a surgical diode laser system (i.e., in the form of a conventional mains-powered surgical diode laser installed in a desktop control panel). The optical wand may incorporate a rolling massage ball facilitating a rolling and kneading action that may increase blood circulation during laser application.

A notable disadvantage with the apparatus described by Pryor et al. is that the wand and pad design render it suitable for topical/skin application only. Not only is the wand design unsuitable for intra-oral applications, but the roller ball can also readily trap dirt, dead skin, dander, and skin microorganisms inside its enclosure, which can pass from patient to patient if it is not disassembled and cleaned after every use.

Specific to the field of dentistry, WO2022/060800 (Cyberdontics USA Inc.) describes an automated system for laser analgesia to accompany the drilling of teeth by a dental drill that is under robotic control. The laser is operating in one or more cycles as needed, to prevent or reduce discomfort caused by the mechanical drilling process. However, the Cyberdontics application fails to provide any description of how the dental drill and laser may be configured to achieve the purported benefits. Indeed, the patent specification is wholly silent on any detail of the laser, drill and housing configuration.

It would be advantageous if there was provided an instrument for effecting PBM therapy on both the skin and mucosa, for use in a wide range of applications, including dental procedures, healthcare settings, cosmetic procedures, domestic settings, and veterinary care. It would also be advantageous if embodiments of the instrument could be used without the need for a standalone high-powered laser source.

### SUMMARY OF INVENTION

In accordance with a first aspect of the present invention there is provided a dental instrument, comprising: a body comprising a light emitting head; and a light source for emitting light through an emitting portion of the light emitting head, the light source configured to simultaneously emit light at multiple peak wavelengths to perform photobiomodulation (PBM) therapy within a patient's oral cavity for accompanying a dental procedure and wherein the multiple peak wavelengths fall within a spectral range of 615 nm to 1100 nm.

In an embodiment the light source is configured to simultaneously emit light at three distinct wavelengths within the spectral range. For performing PBM analgesia, a first of the three distinct wavelengths is near infrared falling within a range from about 900 nm to 1000 nm. The range is preferably about 920 nm to 980 nm. A second one of the distinct wavelengths is also near infrared falling preferably falling within a range from about 800 nm to 910 nm and more preferably from about 820 nm to 860 nm. A third one of the distinct wavelengths may be either visible red or near infrared and preferably falls within a range from about 615 nm to 820 nm.

For analgesia, the light source is preferably controlled such that the first wavelength has a greater intensity than light emitted at the other wavelengths.

In an embodiment the light source comprises an LED broadband emitter.

In an embodiment the light source comprises a plurality of LEDs configured to emit light at the respective peak wavelengths.

In an embodiment the instrument further comprises a controller configured to selectively control at least one of a pulse frequency and pulse energy of the light source sufficient to produce dental analgesia.

In an embodiment the light source is further configured to emit light at wavelengths in the visible and/or red spectrums for performing one or more additional dental functions selected from the group comprising: white light examination; near infrared (NIR) transillumination; fluorescence check; light curing; photocoagulation and photodynamic therapy.

In an embodiment an outer surface of the light emitting head is sealable for preventing ingress of substances present within the patient's oral cavity.

In an embodiment the light source is configured to emit light through a tip of the head.

In an embodiment the body comprises a handle and wherein the light emitting head is connected to the body via an elongate neck, such that the head and at least a portion of the neck are configured for insertion into a patient's oral cavity. At least a portion of the neck and/or light emitting head may be curved or otherwise has a longitudinal axis that is offset with respect to a longitudinal axis of the handle thereby allowing the light emitting head to access posterior areas of the oral cavity. The light source may be integrated into at least one of the head and neck of the instrument and wherein the head/neck is configured to be detachably coupled to the body via an electrical coupling, thereby allowing the light source to be powered and controlled. Alternatively, the light source may be integrated into the body and wherein optical fibre(s) within the neck and head act as light guides for transmitting the emitted light through and out the head. The light source may comprise a cluster of emitters and one or more lenses may be positioned in front of the light source for collimating the light.

The controller may be configured to set a duty cycle for the emitted light and the duty cycle may be set by a user of the instrument.

The controller may be configured to operate the light source(s) in pulsed mode. Pulsing may be achieved by chopping a continuous wave beam emitted by the light source(s).

In an embodiment the emitting portion of the head has an effective diameter of between about 0.2 cm² to 0.8 cm². The dispersion of light emitted from the emitting portion of the head may have a maximum angle of about 10 degrees.

In an embodiment the light source is controlled such that the irradiance for effecting analgesia is between about 8 to 12 Joules per cm².

In accordance with a second aspect there is provided a detachable photobiomodulation (PBM) tip for a handheld dental device having a power supply and controller, the detachable PBM tip comprising: an elongate body having a first end and a second end, the second end configured to electrically and physically couple to a body of the handheld dental curing device; a light source for emitting light through an emitting portion of the light emitting head, the light source configured to simultaneously emit light at multiple peak wavelengths to perform photobiomodulation (PBM) therapy within a patient's oral cavity for accompanying a dental procedure and wherein the multiple peak wavelengths fall within the spectral range of 615 nm to 1100 nm.

In accordance with a third aspect there is provided an instrument configured to perform photobiomodulation (PBM) therapy, comprising: a body comprising a light emitting head; and a light source for emitting light through an emitting portion of the light emitting head, the light source configured to simultaneously emit light at multiple peak wavelengths to perform photobiomodulation (PBM) therapy and wherein the multiple peak wavelengths fall within the spectral range of 615 nm to 1100 nm. The head may be connected to the body via a neck and wherein a portion of the neck and/or head that is curved or otherwise has a longitudinal axis that is offset with respect to a longitudinal axis of the handle.

In accordance with a fourth aspect there is provided a photobiomodulation (PBM) system, comprising: an instrument as described in accordance with any one of the above three aspects and a remote controller configured to wirelessly communicate with the instrument controller for remote control thereof.

In accordance with a fifth aspect there is provided a dental instrument configured to perform photobiomodulation (PBM) therapy, comprising: a body comprising a light emitting head; and a light source for emitting light through an emitting portion of the light emitting head, the light source configured to emit light at one or more wavelengths within the spectral range of 615 nm to 1100 nm to perform photobiomodulation (PBM) therapy within a patient's oral cavity for accompanying a dental procedure and wherein the head is connected to the body via a neck and wherein a portion of the neck and/or head that is curved or otherwise has a longitudinal axis that is offset with respect to a longitudinal axis of the handle allowing easy access to locations with the oral cavity.

In accordance with a sixth aspect there is provided a method of performing a clinical procedure on a human patient or animal using the instrument in accordance with any of the afore-described instruments and such that, in use, the light source is used to irradiate a site on the mucosa or skin prior to administering a local anaesthetic solution using an injection apparatus, to reduce discomfort associated with the injection.

In accordance with a seventh aspect there is provided a method of performing a clinical procedure on a human patient or animal using the instrument in accordance with any of the afore-described aspects and such that, in use, the light source is used to irradiate a site on the mucosa or skin to achieve analgesia, and improve comfort during the procedure.

In accordance with an eighth aspect there is provided a method of performing a cosmetic procedure on a patient or animal using the instrument in accordance with any of the afore-described instruments and such that, in use, the light source is used to irradiate a site on the mucosa or skin for achieving analgesia, improving comfort during a procedure and/or inducing anti-inflammatory effects to manage conditions such as mucositis.

In accordance with a ninth aspect there is provided a method of performing a clinical procedure on a human patient or animal using the instrument in accordance with any of the afore-described instruments and such that, in use, the light source is used to irradiate a temporomandibular joint whether from outside or inside the oral cavity for achieving at least one of the following: analgesia, improving comfort during a dental procedure, relieving spasm; inducing anti-inflammatory effects to manage conditions such as temporomandibular joint dysfunction (TMD) or arthritis.

In accordance with a tenth aspect there is provided a method of performing a clinical procedure on a human patient or animal using the instrument in accordance with any of the afore-described instruments and such that, in use, the light source is used to irradiate a joint or muscle for achieving at least one of the following: analgesia, improving comfort during a dental procedure, relieving spasm; inducing anti-inflammatory effects to manage conditions such as arthritis, muscle fatigue or myositis.

In accordance with an eleventh aspect there is provided a method of performing a clinical procedure on a human patient or animal using the instrument in accordance with any of the afore-described instruments and such that, in use, the light source is used to irradiate facial and/or masticatory muscles, for achieving at least one of the following: analgesia, improving comfort during function, improving mouth opening, relieving spasm and inducing anti-inflammatory effects and symptoms associated with temporomandibular joint dysfunction (TMD), bruxism or animal masticatory myositis.

### BRIEF DESCRIPTION OF DRAWINGS

Embodiments of the present invention will now be described, by way of example only, with reference to the accompanying drawings, in which:

FIG. 1 is a schematic of a therapeutic instrument (in exploded view), in accordance with an embodiment of the present invention;

FIG. 2 is a schematic depicting an example array of emitters implemented by the instrument of FIG. 1;

FIG. 3 is a schematic illustrating an example LED and lens configuration for the instrument of FIG. 1;

FIG. 4 is a schematic of a therapeutic instrument in accordance with an alternative embodiment of the invention;

FIG. 5 is an exploded view of the emitter attachment head of FIG. 4;

FIG. 6 shows respective coupling ends for the instrument of FIG. 4;

FIG. 7 is a table showing emitter wavelengths and corresponding modes of operation; and

FIG. 8 is a graph showing EPT scores for a randomised single-blinded clinical trial.

### DETAILED DESCRIPTION

Embodiments of the invention described herein relate to instruments and methodologies for performing PBM therapy, including effecting PBM analgesia to attenuate injection pain and to establish analgesia, allowing numerous procedures to be performed without the need for prior injection of local anaesthetic solution, topical anaesthetic creams, or other measures to control the pain of the accompanying procedure. It will be understood that the therapy can be performed before, during and/or after the procedure as required. Embodiments also extend to expediting healing and reducing inflammation and swelling after surgery, such as after tooth extraction or periodontal pocket debridement. Other applications include promoting hard tissue formation to seal the pulp or enhance osseointegration between bone and implants.

Embodiments are particularly suited for dental (especially intra-oral) applications and accordingly, the following detailed embodiments will be described in such a context. It will be understood, however, that embodiments can extend to other applications, including for use in healthcare settings, cosmetic procedures, domestic settings, and in veterinary care. For example, the instrument may be configured to irradiate a joint to achieve analgesia, improve comfort during movement and exercise, relieve spasm and induce anti-inflammatory effects and symptoms associated with arthritis. The joint may, for example, be the temporomandibular joint (irradiated either from outside or inside the oral cavity) to relieve symptoms associated with temporomandibular joint dysfunction (TMD). In yet another non-limiting example embodiment, the instrument may be used to irradiate facial and/or masticatory muscles for treating bruxism, masticatory myositis, fatigue and the like.

### Device Configuration

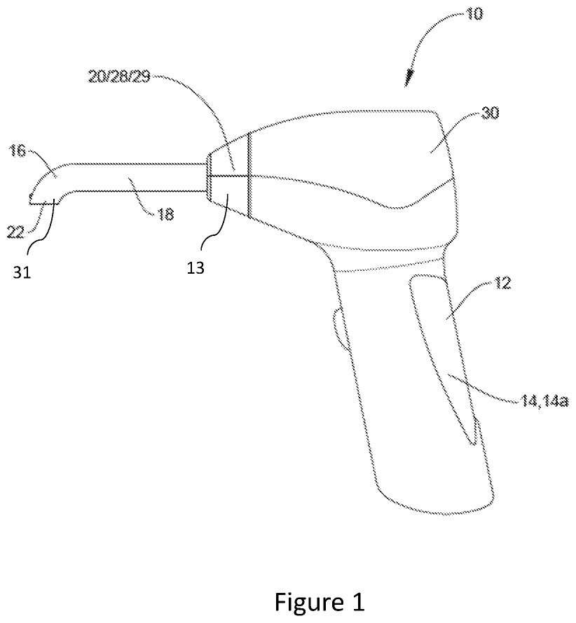

With reference to FIG. 1, there is shown a first embodiment of a therapeutic instrument in accordance with the invention. The therapeutic instrument 10 is lightweight and designed such that it is suited for both skin and intra-oral applications. More particularly, the therapeutic instrument 10 comprises a body 12 having a handle portion 14. According to the illustrated embodiment, the body 12 is formed of any suitable material, such as robust plastic or stainless steel. A housing 14a within the handle portion 14 is suitably configured to receive an integrated electrical power source (not shown), such as a rechargeable battery.

The therapeutic instrument 10 further comprises a head 16 which is connected to the body 12 via an elongate neck 18. According to the illustrated embodiment, the head 16 and at least a portion of the neck 18 are particularly configured for insertion into a patient's oral cavity, e.g., for effecting PBM analgesia as will be described in detail in subsequent paragraphs. In this regard, and as depicted in the figures, a portion of the neck 18 may be curved or otherwise have a longitudinal axis that is offset with respect to a longitudinal axis of the handle portion 14, thus allowing the head 16 to advantageously access posterior areas of the oral cavity and other areas of the body that are difficult to access. In one embodiment, the head 16 may be rotatably coupled to the neck 18 to allow the light-emitting portion of the head 16 (described in subsequent paragraphs) to be directed to hard-to-reach locations. In another embodiment, both the head 16 and neck 18 may be rotatable and coupled to the body 12. In yet another embodiment, the neck 18 may be formed of a flexible material that allows it to be bent into a desired shape.

The therapeutic instrument 10 additionally incorporates a light source 20 that is configured to emit light through an emitting portion 22 of the head 16. Although the illustrated embodiment shows the emitting portion 22 located at the tip of the head 16 (and accordingly will hereafter be referred to as the “emitting tip”), it will be understood that the emitting portion may be located in other regions of the head or the body, depending on the desired implementation. The emitting tip 22 has an effective diameter in the range of approximately 6 to 10 mm, thereby providing an effective light emitting area between approximately 0.2 cm² to 0.8 cm². The dispersion of light from the emitting tip 22 will typically range between 6 to 10 mm. Optical, mechanical and/or digital mechanisms (not shown) may be implemented separately or jointly to control the choice, shape, the diameter of the beam and/or the spot thus decreasing or increasing the effective light emission.

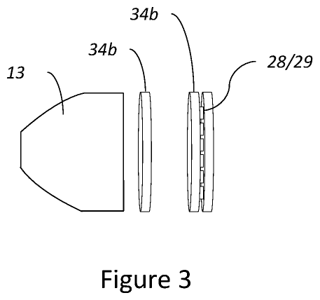

The light source 20 shown in FIG. 1 is integrated into a nose portion 13 of the body 12 (as depicted in more detail in FIG. 3) and comprises at least one light emitter 28 configured to emit light for performing PBM therapy.

According to a preferred embodiment of the invention, the light source is configured to simultaneously emit light at multiple wavelengths (and more preferably three wavelengths) for achieving PBM analgesia or other PBM therapy, as will be described in more detail in subsequent paragraphs.

According to the FIG. 1 embodiment, the neck 18 and head 16 are jointly configured to operate as a light guide for transmitting light emitted from the emitters 28 through the neck 18 and out the emitting tip 22. This may be achieved using any suitable light guide medium, including utilising one or more optical fibres for light transmission. As schematically illustrated in the exploded view of FIG. 3, one or more lenses 34 (in this case two lenses 34a, 34b) are positioned in front of the emitters 28 for collimating the emitted light. In another embodiment, the light source 20 may be configured such that light intensity decreases with distance from the end of the instrument to the target tissue.

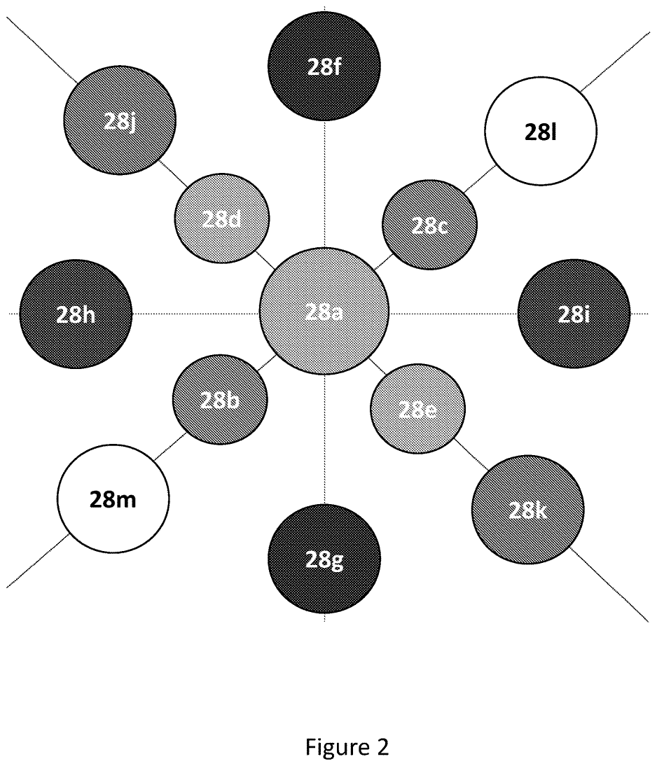

As shown in FIG. 2, an example array of emitters 28a . . . 28n (hereafter “emitter array 29”) may advantageously allow the instrument 10 not only to effect PBM therapy (including PBM analgesia), but also to carry out additional procedures including, but not limited to, white light examination, near infrared (NIR) transillumination, fluorescence checking of decay and tooth-coloured fillings, light curing, photocoagulation and photodynamic therapy.

It will be understood that the emitters 28a to 28m may be disposed in a different arrangement to that shown in FIG. 2, depending on the desired implementation. Table 1 below lists the respective wavelengths of the emitters.

TABLE 1
Emitter Array Table.

| Emitter No. | Wavelength Region | Wavelengths | |
|---|---|---|---|
| 28a | Near Infrared | 860 | nm |
| 28b, 28c | Visible - red | 770 | nm |
| 28d, 28e | Near Infrared | 950 | nm |
| 28f, 28g | Visible - violet | 405 | nm |
| 28h, 28i | Visible - cyan blue | 460 | nm |
| 28j, 28k | Visible - royal blue | 450 | nm |
| 28l, 28m | Visible - white light | 400-700 | nm |

According to Table 1, emitters 28a to 28e are provided primarily for inducing analgesia. It will be understood (and as is evident from the above table) that two or more emitters emitting at the same wavelength may be provided to achieve higher irradiance values.

In an alternative embodiment to that shown in the figures, the light source 20 may comprise a single broadband emitter (e.g., Thorlabs model MBB2D1, manufactured by Thorlabs, Inc: https://www.thorlabs.com/thorproduct.cfm?partnumber=MBB2D1) that is configured to simultaneously emit light at two or more distinct wavelengths. Depending on the desired implementation, the light source may also comprise a combination of broadband and individual wavelength emitters.

A controller 30 is also provided (in this case housed within the body 12) and configured to selectively control the emitters 28 to turn on or off. The controller 30 may be further configured to control the intensity of light emitted by each of the emitters 28. According to embodiments described herein, the controller 30 is configured to power the emitters 28 so that the irradiance ranges between 2 to 12 Joules per cm², depending on the therapy. It will be understood that the relative power and energy output may be controlled by selectively turning on/off emitters 28 of the same wavelength, or by controlling the power provided to each emitter 28 (depending on the instrument and emitter configuration).

The emitters 28 may be pulse-controlled by the controller 30 to minimise heating during operation and to prevent heat accumulation in teeth or tissues that may be discoloured or pigmented, respectively. It will also be understood that emitters 28 may also be pulse-controlled depending on the desired implementation/procedure. According to the illustrated embodiment, pulse control is achieved via modulating pulse width, with the emitters operating in chopped continuous wave mode. A particular advantage of this aspect is that a low-duty cycle can be set for performing various modes of operation (e.g., 25% or 50%), as will be described in more detail in subsequent paragraphs.

The controller 30 may also be configured to communicate with one or more sensors 31 located on or near the head 16 for determining instrument efficacy, as well as for ensuring patient safety. By way of example, a temperature sensor (e.g., infrared temperature sensor) and contact sensor (e.g., optical sensor or capacitance sensor or resistive contact sensor) may be incorporated into the head 16. In this case, where the temperature recorded by the temperature sensor exceeds a predefined maximum, the controller 30 may be programmed to turn one or more of the emitters 28 off for safety reasons. For certain applications (e.g., when used to irradiate a site on the mucosa or skin prior to injection), the controller 30 may be programmed to turn on selected emitters only once the contact sensor indicates that the instrument is in contact with the mucosa/skin.

A sensor may also be used to determine treatment efficacy. For example, as mentioned in preceding paragraphs, the instrument 10 may be used to treat temporomandibular disorders (TMD). In this case, when applied to the joint under treatment, the temperature recorded by a temperature sensor may be used to indicate an elevated temperature when the local blood flow has increased because the surrounding muscle has relaxed, thereby indicating that the treatment is working.

The controller 30 may comprise a microprocessor or the like implementing program code (stored in memory) for performing procedures according to predefined programs (which selectively control emitter operation, intensity, and duration). It will be understood that an interface communicable with the controller 30 may be provided on the instrument to allow an operator to switch between programs or manually control the operation of the emitters 28. In one embodiment, the instrument controller 30 can be further connected, by wires or wirelessly (e.g., via Bluetooth, wi-fi and over the Internet using suitable wireless communications protocols, etc.), to a remote monitor and/or controller (e.g., implemented as a mobile phone device, tablet, PC or the like) to show and/or change the settings remotely.

According to the embodiment shown in FIG. 1, the neck 18 and head 16 are configured to operate as a light guide for transmitting light emitted from the emitter array 29 through the neck 18 and out the emitting tip 22. This may be achieved using any suitable light guide medium, including utilising one or more optical fibres for light transmission. As schematically illustrated in the exploded view of FIG. 3, one or more lenses 34 (in this case two lenses 34a, 34b) are positioned in front of the emitter array 29 for collimating the emitted light. In another embodiment, the light source may be configured such that light intensity decreases with distance from the end of the instrument to the target tissue.

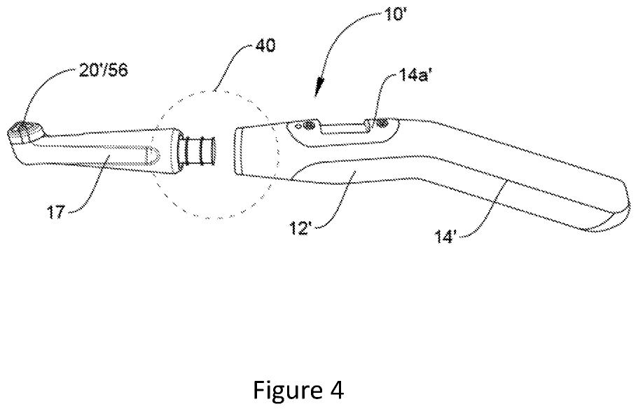

A therapeutic instrument 10′ according to an alternative embodiment of the invention is shown in FIGS. 4 through 6. In this embodiment, the instrument 10′ has a pen-shaped body 12′ with a substantial portion of the neck 18′ lying in generally the same longitudinal plane as the body 12′ (although it will be understood that the shape of the body could be the same as per the embodiment shown in FIG. 1). A notable difference between the illustrated embodiments is that instead of the light source 20′ being incorporated into the instrument body, it is instead located in the neck 18′ and/or head 16′. As for the first embodiment, the light source 20′ comprises either an emitter array, a broadband emitter, or a combination of the two.

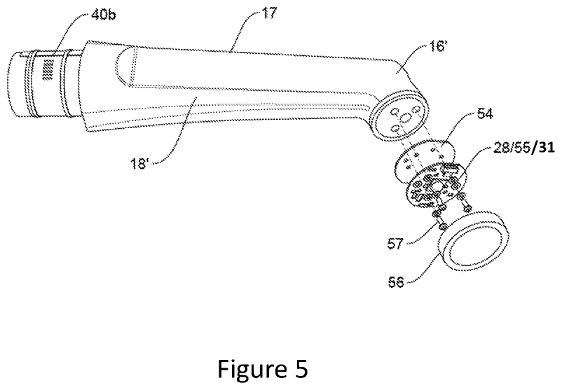

The one or more emitters 28′ are in this case integrated into a multi-component LED panel 55 that is disposed behind an emitting tip 22′ of the head 16′ (this is best shown in FIG. 5). More specifically, the multi-component LED panel 55 contains multiple PBM emitters 28 in the one package, and, depending on the application, can selectively emit light at different wavelengths (including simultaneous light emission at the three distinct peaks described above), which pass from the emitter through a lens 56. The lens 56 is attached, for example, by a screw thread. The LED panel 55 is held in place by fixation screws 57, which attach it to a thermally conductive pad 54 and an aluminium heatsink 53. The body 12′ of the instrument 10′ also serves as a heatsink.

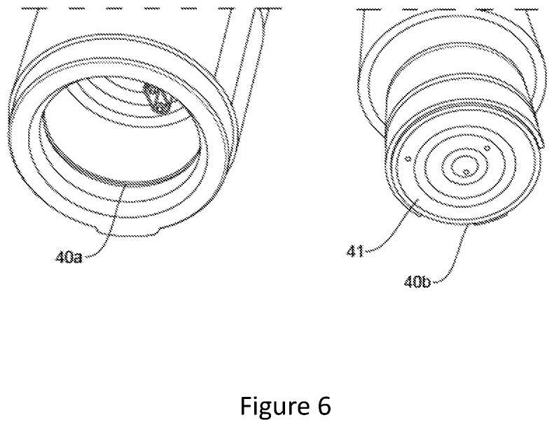

As shown, the head 16′ has an angled design to readily allow PBM therapy (including analgesia) to be delivered in the mouth. According to the illustrated embodiment, the head 16′ and neck 18′ are detachable from the body 12′ and take the form of a replaceable emitter attachment 17. More particularly, the emitter attachment 17 connects to the body 12′ via a suitable coupling 40. The coupling 40 may, for example, be a snap in, plug in or screw in coupling that advantageously allows the light source in the emitter attachment 17 to be powered and controlled by the battery/controller disposed in the body 12′. FIG. 6 shows respective coupling ends for the instrument body and emitter attachment. As shown, the attachment side end includes concentric conductor rings 41 that provide power to the emitters as well as providing a channel for feedback from the light source if required (e.g., if laser diodes are utilised for the light source, a return channel to the controller may be utilised to control deviations in wavelength). An example coupling suitable for use with the present invention is described in U.S. Pat. No. 9,693,846 (Kerr Corporation), the contents of which are incorporated herein by reference. The detachable coupling 40 also allows the body 12′ to power and control a suite of different emitter attachments 17a to 17n (i.e., each having an emitter and/or array of emitters with different wavelength combinations that cover the range as afore described for the first embodiment, thereby allowing the instrument to perform the same operations).

In yet another alternative embodiment (not shown), the emitter attachment 17 may couple to existing equipment, such as to a dental chair, a medical laser or dental laser system, or a powered injecting apparatus. In this case, the existing equipment may be used to power and control the emitter attachment 17 for performing the aforementioned functions. In a particular embodiment, the emitter attachment 17 of the present invention may be configured to electrically and physically couple to a dental curing light designed for photopolymerization of dental materials (such as described in the U.S. Pat. No. 9,693,846). Such curing lights include a rechargeable battery, a control circuit, and a standard attachment incorporating LEDs emitting visible blue light (typically in the 460-480 nm wavelength region). These lights are typically purpose-built as a stand-alone device, sitting on the bench or integrated into a dental chair. Thus, the provision of an emitter attachment 17 with a light source configured to emit light at a wavelength within the range from 615 nm to 1100 nm allows the curing light to additionally operate as an instrument for performing PBM analgesia (as well as any of the other afore-stated operations).

According to any of the afore-described embodiments, an outer surface of the instrument 10, 10′ may be sealed against contamination (e.g., free of any openings or joins through which liquid etc.) for fast and easy cleaning and disinfection. In an embodiment, a disposable sleeve may be fitted over the instrument during use to prevent cross-contamination, and the head 16′ can be removed for sterilisation. Additionally, a window, within or separate from the sleeve, of suitable transparent or translucent material such as sapphire can be removably placed on top of the lens 56 or emitting tip 22, which can be removed after exposure to be sterilised or replaced between patients.

Where the PBM therapy is non-dental, it will be understood that the light source may be incorporated directly into the body or via any suitable coupling (i.e., omitting the elongated neck, which is particularly advantageous for intra-oral applications).

### PBM Modes

In PBM, it is understood that the effects of light are based on the modulation of several metabolic, biochemical, and photophysical processes within cells. Analgesic actions of PBM occur primarily through light interacting with nerves and nociceptors. Reported effects of PBM that cause analgesia include lowered excitability and suppressed electrical responses of nociceptors (pain receptors); lowered hyperpolarisation of neural cell membranes and suppressed nerve responses; elevated compound action potential latencies causing conduction block of nerves; and impaired axonal flow within nerves. However, a more basic issue is that of wavelength optimisation for analgesia.

Regarding penetration, for maximum effectiveness, it is necessary to choose light wavelengths that penetrate deeply into the target tissue and can absorb into the target molecules. For strong transmission, it is known that transmission of light through tissue is highly wavelength-specific. An “optical window” exists in tissue in the approximate range of 500-1200 nm. In this spectral region, there is no major absorber, and hence light delivered onto the surface will penetrate deeply into tissue and scatter widely, especially when the longer wavelengths are used. Light can penetrate to a depth of up to 30 mm depending on the choice of wavelength and the characteristics of the target tissue.

To ensure strong PBM actions for analgesia, it is necessary to choose wavelengths that correspond with the known absorption spectra of the target molecular chromophores (light-absorbing molecules), since peaks in absorption spectra represent regions of increased absorption efficiency.

The present inventor has found that wavelengths ranging from 615 nm to 1100 nm are optimal for PBM actions. More particularly, the inventor has found that simultaneously emitting light at two or more peak wavelengths within this spectrum can provide superior therapeutic effects to conventional single-wavelength techniques. Preferably, emitting light at three peak wavelengths that fall within the spectral range of 615 nm to 1100 nm has been demonstrated to produce effective results for a range of PBM therapies, particularly PBM analgesia.

Preferably, for inducing analgesia, the light dose (total irradiance delivered by the instrument 10) is between 8 to 12 Joules per cm². Further, the light source is preferably configured so that the relative power distribution is weighted toward the higher near-infrared wavelengths which have been demonstrated to be the most effective for inducing analgesia. By way of example, 8 Joules from a 950 nm emitter and 2 Joules respectively for 860 nm and 770 nm emitters (thus resulting in a total light dose of 12 Joules).

At least one of the wavelengths is near infrared preferably falling within a spectral range of between about 900 nm to 1000 nm and more preferably between about 920 to 980 nm. A second one of the wavelengths is also near infrared preferably falling within a spectral range of between about 800 to 910 nm and more preferably between about 820 to 880 nm. Depending on the application, a third one of the wavelengths may be either near infrared or visible infrared falling within the spectral range of between about 615 nm to 820 nm. For PBM analgesia the third wavelength may be toward the higher end of the range (e.g., around 770 nm), whereas for other PBM therapies, such as for muscle relaxation, it may be more desirable for the power distribution to be weighted toward one of the lower wavelengths (such as about 700 nm). From extensive testing, it was found that simultaneously emitting light with wavelengths falling within the aforementioned ranges resulted in effective PBM and penetration through both soft and hard tissues, including bone, dental enamel, and dentine.

The present invention is further described below by way of a non-limiting example.

### Example: PBM for Analgesia

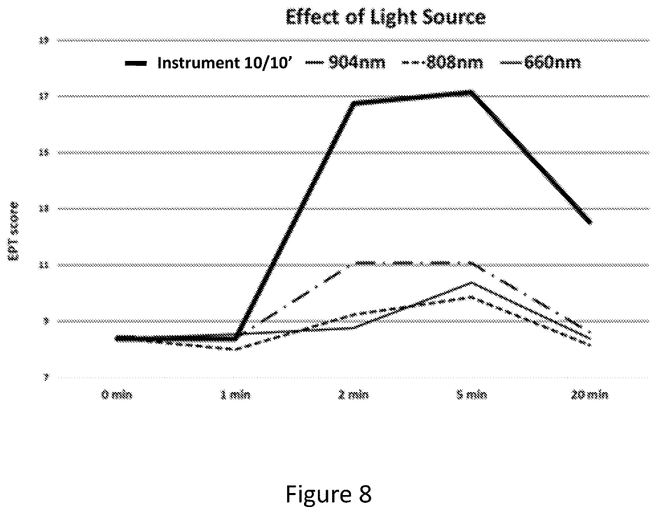

A randomised single-blinded clinical trial was carried out by the inventor as part of their testing. A total of 13 healthy young adults were exposed to PBM therapy using both the subject instrument 10 (operating at three peak wavelengths of about 770 nm, 860 nm and 950 nm) and three commercial diode lasers having wavelengths of 660 nm, 808 and 904 nm respectively. The responsiveness of a subject's dental pulp was quantified using electric pulp testing (EPT), to establish response thresholds. All four light sources were operated with the same spot size of 8 mm and were matched for energy density and total irradiance. A total light dose of 12 Joules was delivered to each subject's premolar tooth from the buccal side and then from the lingual side, and EPT assessments were repeated at 1, 2, 5 and 20 minutes, to assess analgesia from PBM (with an elevation in EPT score indicating analgesia). The trial used a repeated measures design with the same tooth being assessed with all four light sources but on different days. A graph illustrating the resultant EPT score for each light source is shown in FIG. 8. As can be seen, analgesia from the subject instrument 10 was superior to that of the other individual laser light sources, with a larger elevation in EPT scores at 2 minutes, and a longer period of elevated EPT scores compared to other light sources. The analgesic effect resulting from the subject instrument 10 was steady at 5 minutes and declined at around 20 minutes. Other in vitro studies demonstrated that the broadband emissions from the subject instrument 10 penetrated better through teeth of normal shades as well as discoloured teeth, to reach the dental pulp. Furthermore, no subjects experienced any heat or discomfort from the subject instrument 10.

Another clinical trial carried out by the inventor included a responder analysis (showing the extent of change) and data on discomfort caused by the light sources. Again, the trial included testing the subject instrument 10 (operating at the same afore-described three peak wavelengths) against three commercial diode lasers having wavelengths of 660 nm, 808 and 904 nm respectively. This trial included a total of 10 adult subjects (4 male, 6 female) with an age range of between 22 to 63 years. The average age was 30.9 years, and the median age was 24.5 years. A total of 33 teeth were used in a repeated measures design. All teeth were premolars (24 first premolars and 9 second premolars). Site pairing included: 4 subjects with 2 MX and 2 MD premolars (2 matched pairs); 2 subjects with all 4 MX premolars (2 matched pairs); 1 subject with 3 lower premolars (1 matched pair); 2 subjects with 2 MD premolars (not matched); and 1 subject with a single premolar tooth. At the subject level, all 10 subjects showed a positive response to the subject instrument 10.

TABLE 2
EPT elevation achieved in 33 teeth.

| | Non- | By | By | By | By | By |
|---|---|---|---|---|---|---|
| | responder | 25% | 50% | 100% | 150% | 200% |
| 660 nm | 23 | 5 | 4 | 1 | 0 | 0 |
| 808 nm | 24 | 7 | 2 | 0 | 0 | 0 |
| 904 nm | 7 | 5 | 13 | 3 | 2 | 3 |
| Subject | 3 | 3 | 7 | 9 | 2 | 9 |
| Instrument 10 | | | | | | |

As shown in Table 2 above, the response rates at the site level were 30/33 (91%) with an EPT score elevation of at least 25%. Subject instrument 10 was superior to all 3 lasers for actual EPT score change, as well as for percentage change from baseline. The study consisted of 132 experimental runs (33 teeth×4 light sources). The total number of events causing discomfort was 29. None of these resulted from the subject instrument 10, which was demonstrated not to cause any discomfort or sensation in any subject. The discomfort events were mostly with the 808 and 904 nm lasers and were consistent in the same tooth in the same person and were more common in females than males.

An emitter for a suitable visible red wavelength, such as 635 nm or 685 nm, may be powered on while performing PBM analgesia (or other therapies) at the same time as the effective emitters, but at a relatively low intensity to provide a visible indication of proper operation of the instrument 10. With regards to intensity, the controller 30 is configured to control the light source 20 to deliver light onto the target tissue at an intensity that is suitable for gaining PBM actions. The controller 30 may also control the light source for illuminating the site in a desirable manner when visible red and near infrared light are used for the transillumination of soft tissues or hard tissues for purposes of clinical examination.

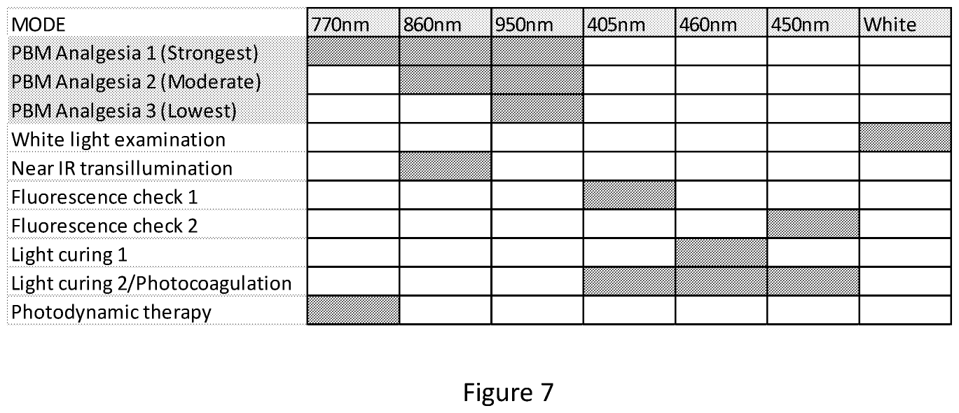

With reference to the table shown in FIG. 7, the controller 30 may selectively control each emitter 28a . . . 28n either alone, or in combination with other emitters, to perform the therapeutic operations. It will be understood from the table that turning on multiple emitters simultaneously can be used to either enhance therapy or perform simultaneous functions. For example, for performing a first preferred mode of analgesia (PBM Analgesia 1, which has the greatest analgesia effect), the controller 30 will turn on the 950, 860 and 770 nm emitters (for reasons as described above). A second mode of analgesia (PBM Analgesia 2, having a moderate analgesia effect) is performed by the controller turning on both the 950 nm and 860 emitters. For performing PBM Analgesia 3 (lowest analgesia effect), the controller 30 will turn on the 950 nm emitter only.

### Examples of PBM Operation Modes:

The following describes in detail four indicative applications in clinical dental practice. For each of these four situations, the emitting tip 22, 22′ has a terminal effective diameter of 8 mm, providing an area of 0.50 square centimetres. To provide irradiation with light in contact mode, the emitting tip 22, 22′ is positioned against the target tooth or oral soft tissue and held still by hand using light pressure. A disposable transparent sleeve covers the tip to prevent contamination with saliva or other fluids. In the following examples, the dispersion of light from the emitting tip 22, 22′ has a maximum angle of 10 degrees. In these examples, the irradiance used for analgesia is approximately 10 Joules per square centimetre (though, as stated in preceding paragraphs, the irradiance for the 950 nm wavelength may be greater than the lower wavelengths, e.g., by turning on multiple 950 nm emitters, depending on the mode of treatment). The controller 30 is configured to operate the emitters 28a to 28e in pulsed mode at a frequency of 50 Hz and with a duty cycle of less than 100%. According to the embodiments described herein, the duty cycles may be set to either 25% or 50%. As previously discussed, the lower duty cycle advantageously minimises heating of the light emitters 28a to 28e during operation and prevents heat accumulation in teeth or tissues that may be intensely discoloured or heavily pigmented, respectively. The choice of a lower duty cycle of 25% may be used when the target tooth or site is discoloured or pigmented, respectively. For example, a duty cycle of 25% may be used for patients with dark teeth/mucosa/skin. Avoidance of using continuous wave mode and superpulsed modes prevents excessive heating of superficial regions and deep tissue locations, respectively, to ensure that the irradiation procedure will be free of thermal effects and thus will be painless for the patient. It will be understood that duty cycles other than 25% and 50% may be used depending on the desired application and individual patient circumstances.

With a total optical power of 1.0 watt delivered through the emitting tip 22, 22′ for each wavelength, the effective power delivered to the tissue at a duty cycle of 25% or 50% is either 0.25 or 0.5 watts for each wavelength, hence a fluence of 10 Joules per square centimetre can be achieved in a suitably convenient exposure time of 20 seconds (at 25% duty cycle) or in 10 seconds (at 50% duty cycle), for each site or tooth.

In a first application, prior to commencing scaling of the teeth to remove calcified deposits with hand operated or powered scaling devices, the tooth and the adjacent soft tissues are treated with the instrument 10, 10′ to reduce discomfort during the procedure, by elevating the threshold at which the dental pulp and nociceptors in the soft tissues will respond to external stimuli. Each tooth that is sensitive to compressed air from a dental triple syringe is treated before the procedure using PBM Analgesia 2 with the light directed towards the dental pulp through the area of exposed sensitive dentine. The gingival tissues are treated using PBM Analgesia 3 with the emitting tip 22, 22′ moving slowly at 1-2 mm per second to cover the area where the scaler will be used.

In a second application, prior to commencing removal of decayed tooth structure using hand instruments in the Atraumatic Restorative Technique (a technique initially promoted by the World Health Organisation to deliver dental care in developing countries without access to sophisticated dental treatment, but later developed to more widely applicable Interim Therapeutic Restorations adopted by the American Academy of Paediatric Dentistry), the tooth is treated with the device using PBM Analgesia 1 to attenuate sensations that occur when the vital inner healthy dentine of the tooth is reached during the decay removal process. If discomfort develops during decay removal, a further dose can be applied through the overlying bone and soft tissue to reach the dental pulp. The tooth with its prepared cavity is then treated a second time with the instrument 10, 10′ using the same setting, so that the procedures of rinsing the cavity and placing the filling material can be undertaken with minimal discomfort. At the end of the procedure when the filling has been placed and the bite adjusted, the tooth is treated a final time using PBM Analgesia 3, to reduce the probability of postoperative discomfort. In this setting, the 950 nm provides the PBM effect. Also, visible light from other emitters in the apparatus can be used to help cure, set, or solidify the restorative material of choice.

In yet another application, the injection site for a mandibular block injection is treated with the instrument 10, 10′ immediately prior to administering the injection, with PBM Analgesia 3, to reduce the pain of the needle piercing through the mucosal soft tissues.

In a further application, the site where a clamp will be affixed to a tooth caused by the compression of the tooth itself and the displacement of the adjacent gingival soft tissues. The clamp is then used to stabilise the dental dam prior to restorative or endodontic dental procedures. Alternatively, a clamping device can also be used to attach rigidly onto the tooth surface the intra-oral component of a laser dental drill, allowing the tooth to be treated. The tooth to be clamped is treated immediately before the clamp is placed using PBM Analgesia 2 with the light directed through the tooth crown towards the dental pulp. If the tooth already has a large restoration, such as a full crown, PBM Analgesia 1 is used with the light applied through the overlying bone and soft tissue to reach the dental pulp. The gingival tissues are treated using PBM Analgesia 3 with the tip moving slowly at 1-2 mm per second to cover the area where the clamp will be compressing the gingival tissues.

In yet another application, a site, intra or extra orally, may advantageously be irradiated using one or a combination of the various PBM Analgesia or Therapy modes shown in FIG. 7 before, during and/or after the procedure, e.g. to enhance perfusion, reduce inflammation, reduce swelling, mitigate postoperative pain, as well as to promote healing, hard/soft tissue regeneration and osseointegration with implants.

Whilst the invention has been described above in the context of providing PBM therapy to humans, the invention is not limited thereto and embodiments extend to the provision and use of appropriately configured devices as methods as described herein with animals.

Various advantages arise through one or more of the afore-described embodiments including, but not limited to:
Ensure strong activation of key targets of PBM located in the mitochondria of cells through use of LEDs at optimised wavelengths ranging from 615 to 1100 nm;
Provide a universally applicable simple instrument which may reduce or eliminate the need for topical or injected anaesthetic agents, in healthcare settings, in cosmetic procedures, in domestic settings, and in veterinary care;
Can be provided as either a stand-alone instrument, or as an emitter attachment that can be detachably coupled to existing equipment, such as a dental curing light, dental chair, a medical or dental laser system, or a powered injecting apparatus;
Allow for the delivery of light either along or away from the axis of the long axis of the device, including at right angles thereby making delivery to the mouth and other body regions that are difficult to access possible;
Provide an instrument that is lightweight, handheld and portable, with a simpler and more robust design that existing PBM devices;
Provide an instrument that is suited to addressing issues of infection control, since the entire device can be easily encased in a disposable sheath, and the surface can be wiped down with a detergent/disinfectant product for decontamination;
Provide a universal instrument that is suitable for use with humans and nonhuman animals;
Be applicable to a range of different applications, including for use in healthcare settings, cosmetic procedures, domestic settings, and in veterinary care due to features including variable modes of action, portability, compactness, lightweight body and use of battery power.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The preceding description is provided in relation to several embodiments which may share common characteristics and features. It is to be understood that one or more features of any one embodiment may be combinable with one or more features of the other embodiments. In addition, any single feature or combination of features in any of the embodiments may constitute additional embodiments.

In addition, the foregoing describes only some embodiments of the inventions, and alterations, modifications, additions and/or changes can be made thereto without departing from the scope and spirit of the disclosed embodiments, the embodiments being illustrative and not restrictive.

Furthermore, the inventions have described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the inventions. Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

1-68. (canceled)

69. An instrument for performing pre-emptive photobiomodulation (PBM) analgesia preceding or accompanying a medical or dental procedure, comprising:
a body comprising a light emitting portion; and
a light source configured to emit light through the light emitting portion to target a site on the skin or mucosa of an animal, including a human, that is to undergo the pre-emptive photobiomodulation (PBM) analgesia, the light source configured to simultaneously emit light at multiple distinct peak wavelengths falling within a spectral range of 615 nm to 1100 nm with at least one of the distinct peak wavelengths falling within a near-infrared spectral range of between about 900 nm to 1100 nm and at least one other one of the distinct peak wavelengths falling within a spectral range of between about 615 nm to 900 nm.

70. The instrument in accordance with claim 69, wherein the light source is further configured such that a relative power distribution for the emitted light is weighted toward one or more longer ones of the wavelengths within the spectral range.

71. The instrument in accordance with claim 69, wherein the light source is further configured such that a radiant exposure of light delivered to the target tissue falls between about 8 to 12 Joules per cm².

72. The instrument in accordance with claim 69, wherein the light source is configurable by an operator to dynamically adjust at least one of an emitted power, peak wavelength power distribution, and pulse modulation for achieving a desired level of PBM analgesia.

73. The instrument in accordance with claim 69, wherein the light source comprises a multi-wavelength emitter.

74. The instrument in accordance with claim 69, wherein the light source comprises a plurality of LEDs configured to emit light at the respective peak wavelengths.

75. The instrument in accordance with claim 69, further comprising a controller configured to selectively control at least one of a pulse frequency and pulse energy of the light source sufficient to produce pre-emptive PBM analgesia.

76. The instrument in accordance with claim 75, wherein the controller is configured to set a duty cycle for the emitted light.

77. The instrument in accordance with claim 75, wherein the controller operates the light source(s) in pulsed mode and preferably wherein pulsing is achieved by electrical current pulse, or chopping a continuous wave beam emitted by the light source(s).

78. The instrument in accordance with claim 69, wherein, for dental applications, the light emitting portion has an effective area of between about 0.2 cm² to 0.8 cm².

79. The instrument in accordance with claim 69, further comprising a temperature sensor and display means configured to output a temperature reading.

80. The instrument in accordance with claim 69, wherein the light emitting portion is disposed on a head which is in turn connected to the body via an elongate neck, such that the head and at least a portion of the neck are configured for insertion into the patient's oral cavity for administering pre-emptive PBM dental analgesia.

81. The instrument in accordance with claim 80, wherein at least a portion of the neck and/or light emitting head is curved or otherwise has a longitudinal axis that is offset with respect to a longitudinal axis of the handle thereby allowing the light emitting head to access posterior areas of the oral cavity.

82. The instrument in accordance with claim 80, wherein the light source is further configured to emit light at wavelengths in the visible and/or red spectrums for performing one or more additional dental functions selected from the group comprising: white light examination; near infrared (NIR) transillumination; fluorescence check; light curing; photocoagulation and photodynamic therapy.

83. The instrument in accordance with claim 80, wherein the light source is integrated into at least one of the head and neck of the instrument and wherein the head/neck is configured to be detachably coupled to the body via an electrical coupling, thereby allowing the light source to be powered and controlled.

84. The instrument in accordance with claim 80, wherein the light source is integrated into the body and wherein optical fibre(s) within the neck and head act as light guides for transmitting the emitted light through and out the head for targeted delivery to the site.

85. The instrument in accordance with claim 69, wherein an outer surface of the instrument comprises a contamination barrier.

86. The instrument in accordance with claim 85, wherein the outer surface is sealable.

87. The instrument in accordance with claim 85, further comprising a protective sleeve configured to at least partially cover the outer surface to thereby operate as the contamination barrier.

88. A pre-emptive photobiomodulation (PBM) analgesia system, comprising:
an instrument for performing pre-emptive photobiomodulation (PBM) analgesia preceding or accompanying a medical or dental procedure, comprising: a body comprising a light emitting portion; and a light source configured to emit light through the light emitting portion to target a site on the skin or mucosa of an animal, including a human, that is to undergo the pre-emptive photobiomodulation (PBM) analgesia, the light source configured to simultaneously emit light at multiple distinct peak wavelengths falling within a spectral range of 615 nm to 1100 nm with at least one of the distinct peak wavelengths falling within a near-infrared spectral range of between about 900 nm to 1100 nm and at least one other one of the distinct peak wavelengths falling within a spectral range of between about 615 nm to 900 nm; and
a remote controller configured to wirelessly communicate with the instrument or device controller for remote control thereof.

89. A method of administering pre-emptive photobiomodulation (PBM) analgesia comprising:
providing an instrument for performing pre-emptive photobiomodulation (PBM) analgesia preceding or accompanying a medical or dental procedure, comprising: a body comprising a light emitting portion; and a light source configured to emit light through the light emitting portion to target a site on the skin or mucosa of an animal, including a human, that is to undergo the pre-emptive photobiomodulation (PBM) analgesia, the light source configured to simultaneously emit light at multiple distinct peak wavelengths falling within a spectral range of 615 nm to 1100 nm with at least one of the distinct peak wavelengths falling within a near-infrared spectral range of between about 900 nm to 1100 nm and at least one other one of the distinct peak wavelengths falling within a spectral range of between about 615 nm to 900 nm; and
controlling the light source to emit light to the target site for administering pre-emptive photobiomodulation (PBM) analgesia.

90. The method in accordance with claim 89, controlling the light source such that the radiant exposure of light delivered to the target tissue falls between about 8 to 12 Joules per cm².
